(19) United States
(12) Patent Application Publication
(10) Pub. No.: US 2025/0339057 A1
(43) Pub. Date: Nov. 6, 2025

(54) CONDUCTIVE HYDROGEL-BASED STRAIN SENSORS FOR MEASURING BODY MOVEMENTS

(71) Applicant: Nano and Advanced Materials Institute Limited, Hong Kong (HK)

(72) Inventors: Yuechen WANG, Hong Kong (HK); Ling ZHAO, Hong Kong (HK); Kai Man LAM, Hong Kong (HK); Chun Wah CHU, Hong Kong (HK); Li FU, Hong Kong (HK); Chenmin LIU, Hong Kong (HK)

(21) Appl. No.: 18/655,362

(22) Filed: May 6, 2024

(51) Int. Cl. A61B 5/11; A61B 5/00; B32B 5/02; B32B 5/18; B32B 15/02; B32B 15/06; B32B 15/095; B32B 25/10; B32B 25/20; B32B 27/08; B32B 27/12; B32B 27/40; C08J 3/075; C08J 3/20; C08K 3/04

(52) U.S. Cl. CPC A61B 5/1126; A61B 5/6801; B32B 5/024; B32B 5/18; B32B 15/02; B32B 15/06; B32B 15/095; B32B 25/10; B32B 25/20; B32B 27/08; B32B 27/12; B32B 27/40; C08J 3/075; C08J 3/20; A61B 2562/0261; A61B 2562/164; B32B 2262/103; B32B 2264/108; B32B 2266/0221; B32B 2266/0257; B32B 2266/0278; B32B 2266/122; B32B 2307/202; B32B 2307/51; C08J 2329/04; C08J 2333/26; C08J 2429/04; C08J 2433/26; C08K 3/041; C08K 2201/001

(57) ABSTRACT

A dual mode conductive hydrogel-based strain sensor is provided, which includes both ion conductive mechanism and electron conductive fillers. The hydrogel-based strain sensor includes a hydrogel layer with a first cross-linked hydrogel-forming polymer network and a second cross-linked hydrogel-forming polymer network. The second cross-linked hydrogel-forming polymer network interpenetrates into the first hydrogel-forming polymer network without cross-linking between the two networks. A water-based liquid is entrained by the first and second crosslinked hydrogel-forming polymer networks in an amount of approximately 50-75 wt % of the hydrogel. The water including an ionically-conducting salt in an amount of 5-25 wt % of the formed hydrogel. Conductive fillers include two or more of graphene, carbon nanotubes, and MXene. Stretchable conductive electrodes formed on the hydrogel layer and are selected from conductive particle-filled elastomers, stretchable metal meshes, and stretchable conductive fabrics.

### FIELD OF THE INVENTION

The present invention relates to conductive hydrogel-based strain sensors, in general, and, more particularly, to hydrogel-based strain sensors that are skin-mountable for measuring body movements.

### BACKGROUND

Conventional strain sensors use metal strips that are affixed to a test specimen. As tensile forces are applied to the test specimen, the electrical resistance of the metal strips changes as the metal strips are elongated. This change in electrical resistance is used to determine the test specimen strain. While these strain sensors are adequate for measuring strain in objects that do not undergo extensive deformation, such as a length of structural steel or an aircraft wing component, such strain sensors cannot be used to record anatomical movements of body parts or internal organs where changes in length of a body part can be very large over the course of movement of an appendage such as an arm, leg, or finger.

As a result, hydrogel-based strain sensors have been proposed. Hydrogels, being substantially more elastic than conventional strain sensors, can extend and contract along with the motion of an arm or leg to which the hydrogel is attached. As used herein, the term “hydrogel” relates to three-dimensional hydrophilic polymer networks of hydrophilic polymers that trap least 10% water of the total volume of the hydrogel. Because hydrogels can be made to be extremely elastic and flexible, they can stretch and contract in ways similar to human skin, making them compatible with mounting to the human body.

Prior art hydrogel strain sensors have been proposed in CN115607106A, CN115558229A, and CN113787800A. However, the materials of these strain sensors are not sufficiently sensitive for measurement of very small resistance changes or do not have sufficiently flexible electrodes for incorporation into a practical, wearable strain sensor.

Thus, there is a need in the art for improved hydrogel-based strain sensors; such improved sensors could be used for wearable sensors for measuring body movements.

### SUMMARY OF THE INVENTION

A dual mode conductive hydrogel-based strain sensor is provided having both an ion conductive mechanism and electron conductive fillers. The hydrogel-based strain sensor includes a hydrogel layer having a first cross-linked hydrogel-forming polymer network and a second cross-linked hydrogel-forming polymer network. The second cross-linked hydrogel-forming polymer network is interpenetrated with the first hydrogel-forming polymer network without cross-linking between the first and second hydrogel-forming polymer networks. A water-based liquid is entrained by the first and second cross-linked hydrogel-forming polymer networks in an amount of approximately 50-75 wt % of the hydrogel, the water including an ionically-conducting salt in an amount of 5-25 wt % weight percent of the formed hydrogel. Conductive fillers include two or more of graphene, carbon nanotubes, and MXene. Stretchable conductive electrodes formed on the hydrogel layer and are selected from conductive particle-filled elastomers, stretchable metal meshes, and stretchable conductive fabrics.

In a further aspect, the first hydrogel-forming crosslinked polymer network includes a polyvinyl alcohol-based polymer and the second hydrogel-forming crosslinked polymer network includes an acrylamide-based polymer or a urethane-based polymer.

In a further aspect, the salt is selected from NaCl, CaCl₂, LiCl, or KCl.

In a further aspect, a protective layer is formed over the hydrogel, for example a protective silicone or polyurethane layer.

A wearable flexible strain measurement device may be connected to the dual mode conductive hydrogel-based strain sensor.

The present invention also provides a method for making the dual mode conductive hydrogel-based strain sensor. A mixture is formed from a water-soluble synthetic polymer for the first hydrogel-forming polymer network, a polymerizable monomer for forming the second hydrogel-forming polymer network, and an ion conductive salt solution. The electron conductive filler is added to the first mixture, followed by adding a crosslinking to crosslink the polymerizable monomer. The created hydrogel has an interpenetrating network of the first hydrogel-forming polymer network and the second hydrogel-forming polymer network. A sensor blank is cut from the formed hydrogel. Electrodes are formed on the sensor blank.

In a further aspect, an initiator is added to form the second hydrogel-forming polymer network.

In a further aspect the mixture includes:
0.5-15 wt % of the water-soluble synthetic polymer for forming the first hydrogel-forming crosslinked polymer network;
5-20 wt % of the polymerizable monomer;
5-25 wt % of salt, contributing to the ion conduction;
0.005-3 wt % of the electron conductive filler;
50-75 wt % of deionized water;
the crosslinking agent;
an initiator for a sol-gel process; and
an accelerator for a sol-gel process.

The crosslinking agent may be N,N′-methylenebisacrylamide, the initiator may be ammonium persulfate and the accelerator may be N,N,N′, N′-tetramethylethylenediamine.

### BRIEF DESCRIPTION OF THE DRAWINGS

FIG. 1 is a schematic depiction of a conductive hydrogel according to an embodiment;

FIG. 2 is a strain sensor using the conductive hydrogel of FIG. 1;

FIG. 3 is strain sensor connected to a strain sensor measurement device and transmission circuit; and

FIG. 4 shows a plot of strain sensor resistance vs. time.

### DETAILED DESCRIPTION:

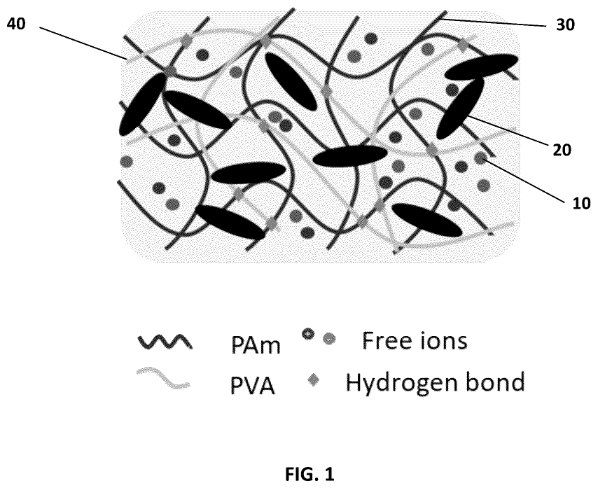

Turning to the drawings in detail, FIG. 1 schematically depicts a hydrogel 100 having mechanical properties that mimic human skin while being sufficiently conductive for incorporation into a strain sensor. By mimicking human skin elasticity, a strain sensor including hydrogel 100 is able to accurately reflect the motion of various human appendages. In order to create an elastic hydrogel, the hydrogel 100 of the present invention uses two polymer networks 30 and 40. Each polymer network may be a crosslinked polymer network; however, polymer networks 30 and 40 are not crosslinked to each other. That is, the two polymer networks are interpenetrated with each other by physical bonded to each other, forming an interpenetrating polymer network. As seen in hydrogel 100, first polymer network 30 and second polymer network 40 interact via hydrogen bonding. Due to the introduced hydrogen bonding, the hydrogel network can easily be stretched by breaking the hydrogen bonds, and recover by re-forming the hydrogen bonds. The reversible physical bonds enable the hydrogel materials to sensitively deform with both small and large strain rates. This more closely mimics the action of human skin, which is stretchable in multiple directions in a single body movement, but readily returns to an unstretched condition at rest.

The first and second polymer networks 30 and 40 can be formed from one or more synthetic polymers including polyacrylate, polyvinyl alcohol, polyethylene glycol, methacrylate-based polymers (e.g., hydroxy methacrylate), polyvinylpyrrolidone, acrylamide polymers, polyurethane. The first and second polymer networks 30 and 40 can also be formed from one or more natural polymers including chitosan, polysaccharides, alginate, gum, pectin, and collagen. In the examples below, the polymers for networks 30 and 40 include polyvinyl alcohol and polyacrylamide; however, it is understood that other polymers, such as the ones set forth above, may also be selected for hydrogel 100.

The water content of the hydrogel is further selected such that the hydrogel mimics the properties of human skin. Typically, a water content of 50-75 wt. % is selected (human skin is approximately 70 percent water). The high water content of the hydrogel also contributes to its sensitive electrical properties. In some embodiments, the water may include an ionically-conducting salt in an amount of 5-25 wt. % the formed hydrogel.

In order to form hydrogel 100 into a strain sensor, conductive additives are incorporated into the hydrogel matrix. Hydrogel 100 is a dual mode conductive hydrogel that includes both ions 10 and conductive particles 20. Together, the ions and conductive particles, create a hydrogel that demonstrates a change in hydrogel resistance even for subtle or small body/muscle movements. Further, a strain sensor made from hydrogel 100 exhibits fast recovery time, which is important for dynamic body movement measurements.

Ions 10 are incorporated into the hydrogel 100 matrix through addition of one or more salts during the hydrogel formation process. These salts include sodium chloride, potassium chloride, lithium chloride, calcium chloride. Conductive particles 20 are selected from one or more of carbon-based materials (e.g., graphite, graphene, carbon nanotubes, KJ black, super P), MXenes (that is a transition metal carbide, nitride, or carbonitride in the form of atomically-thin layers), metal nanoparticles (Au nanoparticles, Ag nanoparticle, Cu nanoparticle), conductive polymers (e.g., polyacetylene (PA), polyaniline (PANI), polypyrrole (PPy), polythiophene (PTH), poly (para-phenylene) (PPP), poly (phenylenevinylene) (PPV), and polyfuran (PF), PE-DOT: PSS). Together, the resistivity of the hydrogel can be selectively tuned to be between low resistivity of a few hundred ohms to resistivity on the order of mega-ohms.

Formation of hydrogel 100 ensures correct formation of polymer networks 30 and 40 and adequate dispersion of the conductive particles throughout the polymer networks for homogenous electrical properties in the hydrogel. In one aspect, this formation can be accomplished by dissolving one water-soluble polymer that forms a first polymer network followed by adding a cross-linkable monomer, ion-conducting salt and conductive particles. In this manner, the conductive particles are more readily dispersed through the mixture. Only after ensuring a homogenous mixture is the monomer crosslinked, resulting in the structure depicted in FIG. 1.

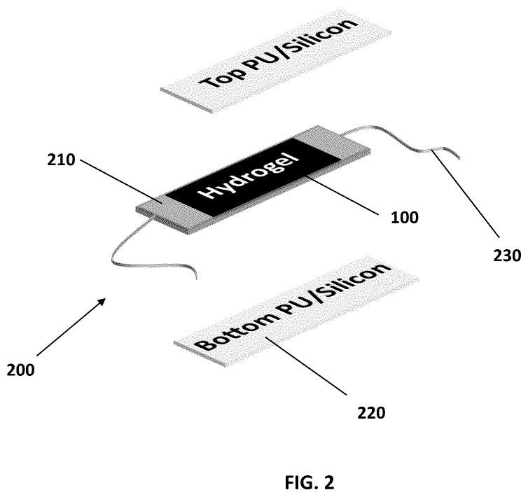

FIG. 2 depicts a strain sensor 200 that includes the hydrogel 100 of FIG. 1. Importantly, the strain sensor 200 includes electrodes 210 that can also flex with the movement of the hydrogel when the sensor is affixed to a human body part for having movement measured. In order to ensure sufficient movement, electrodes 210 may be formed from a conductive mesh or a conductive elastomeric material. Examples include elastomers such as rubber loaded with conductive particles, metal meshes in elastomeric fabrics (e.g., silver or copper mesh embedded in elastomeric fabrics), conductive elastomeric yarns woven into a stretchable structure, etc. In general, it is desirable if the electrodes are soft enough, can adhere to the surface of hydrogel sensing material without delamination and with a certain mechanical property to deform with the whole sensor patch according to the movement on human body.

The strain sensor 200 may optionally include one or more protective layers 220 for protecting the hydrogel 100. Protective layers 220 may be an elastomer such as polyurethane, silicone, PDMS, TPU and SEBS. Leads 230 extend from electrodes 210 for connection to a strain sensor reading circuit.

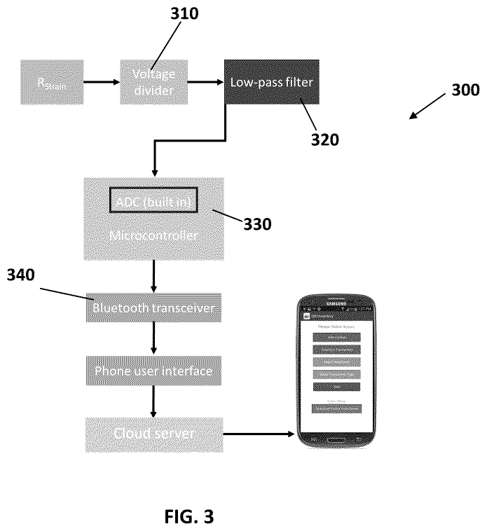

FIG. 3 depicts a system-level overview of signal transduction, conditioning, processing, and wireless transmission paths to facilitate multiplexed on-body measurements. As seen in FIG. 3, the signal processing system 300 includes a voltage divider 310, low pass filter 320, microcontroller 330 with an analogue to digital converter, and a wireless transceiver 340, for example, a Bluetooth transceiver.

The signal conditioning path for each sensor is implemented with analogue circuits and in relation to the corresponding transduced signal. The circuits are configured to ensure that the final analogue output of each path is finely resolved while staying within the input voltage range of the analogue-to-digital converter. Furthermore, the microcontroller's computational and serial communication capabilities are used to calibrate, compensate, and relay the conditioned signals to an on-board wireless transceiver. The transceiver facilitates wireless data transmission to a Bluetooth-enabled mobile handset with a custom-developed application containing a user-friendly interface for sharing or uploading the data to cloud servers.

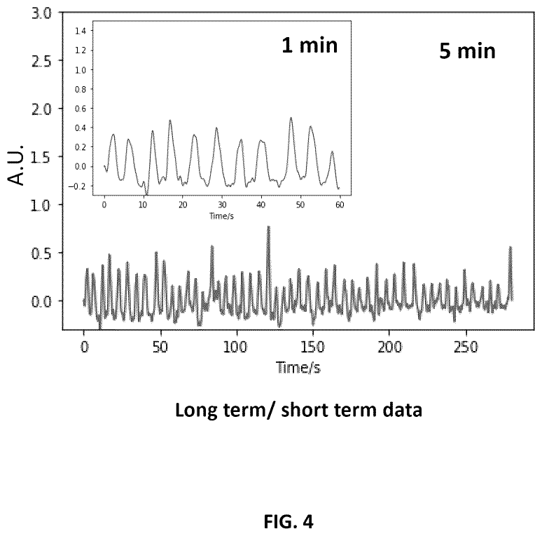

The signals are received by a wireless receiver that is part of a signal processor such as a computer or mobile device. The strain gauge resistance vs. time raw data that is received by the computer is depicted in FIG. 4. The mechanical signal from the movements is converted to electrical signals, movements of body parts create deformations of the strain sensor, which change the distance between the conductive particles. As the distance between the conductive particles changes, the hydrogel resistance changes as shown in FIG. 4.

The present invention also relates to a dual mode conductive hydrogel formation method. The fabrication method of the hydrogel follows the following overall flow:
(S1) A step for dissolving the water-soluble synthetic polymer in deionized water at an elevated temperature between 60-90° C. (e.g., 85° C.) for four hours utilizing magnetic stirring;
(S2) A step for dissolving the monomer for further copolymerization utilizing magnetic stirring;
(S3) A step for dissolving the ion conductive salt utilizing magnetic stirring;
(S4) A step for mixing the electron conductive filler into the as prepared hydrogel solution utilizing planetary-mixer method;
(S5) A step for dissolving the crosslinking agent utilizing magnetic stirring;
(S6) A step for dissolving the initiator utilizing magnetic stirring;
(S7) A step for dissolving the accelerator utilizing magnetic stirring;
(S8) A step for degassing the as prepared hydrogel solution through vacuum drying chamber;
(S9) A step for pouring the degassed hydrogel solution into a glass substrate with 1 mm thick silicon spacer for sol-gel process;
(S10) A step for cut the hydrogel sample into designed size and pattern utilizing laser cutting;
(S11) A step for connecting the flexible silver-plated woven strips electrodes onto the hydrogel;
(S12) A step for encapsulate the Hydrogel with electrodes connected;
(S13) A step for connecting the hydrogel-based strain sensor to a FPCB circuit.

Typical ranges for forming the hydrogel are set forth below:

The dual conduction mode hydrogel sensor is made from a mixture of precursors:
0.5-15 wt % of the water-soluble polymer for forming the first hydrogel-forming crosslinked polymer network;
5-20 wt % of the polymerizable monomer;
5-25 wt % of salt, contributing to the ion conduction, and 0.005-3 wt % of the
electron conductive filler;
the crosslinking agent;
an initiator for a sol-gel process; and
an accelerator for a sol-gel process.

The crosslinking agent may be N,N′-methylenebisacrylamide, the initiator may be ammonium persulfate and the accelerator may be N,N,N′, N′-tetramethylethylenediamine.

### Example 1

Synthesis of the dual mode conductive hydrogel with CNT filler

1.2g of poly (vinyl alcohol) is dissolved in 20 ml of DI water at 85° C. utilizing magnetic stirring for few hours. After 4.69 g of acrylamide is further dissolved in the solution at room temperature for two hours utilizing magnetic stirring, 3.45 g of sodium chloride is added into the solution and magnetic stirred for one hour until its totally dissolved. 10 ml CNT solution poured into the as prepared hydrogel solution for homogeneous dispersion utilizing planetary-mixer at 900 rpm for 5 minutes. 0.007 g of N,N′-Methylenebisacrylamide is dissolved into the mixture solution as a crosslinking agent through magnetic stirring under room temperature. After dissolving 0.02 g of the ammonium persulfate, which acts as the initiator for sol-gel process, 8 μL of accelerator, N,N,N′,N′-tetramethylethylenediamine, is dropwise-added into the solution to form a hydrogel precursor solution. Both the initiator and the accelerator are homogeneously mixed utilizing magnetic stirring under room temperature. The hydrogel precursor solution is poured into a glass substrate with 1 mm silicon spacer. A top layer of glass sheet is covered onto the hydrogel precursor solution for the sol-gel process and control the thickness of the hydrogel sheet. The as-made sensor is extremely sensitive to subtle forces and stretches applied to the human body. It is capable of detecting signals even with full body movement, within a strain rate of 0-75%. The sensor's sensitivity is of GF≥10, and it has a recovery speed of less than 50 milliseconds.

### Conductive Fillers

The conductive fillers include graphene, MXene and CNT. The single layer graphene water dispersion is provided by XFNANO with various concentrations. The MXene water dispersion indicates the Ti₃C₂ with various concentrations and is provided by Beike 2D materials Co., Ltd. The CNT water dispersion is provided by XFNANO utilizing multi-wall CNTs with various concentrations.

### Flexible Silver-Plated Woven Fabric Electrodes

The silver-plated woven fabric is made from 100% silver fiber, with >99.9% bacteriostasis rate, <1Ω/S surface resistance and >55 DB shielding efficiency. The one-piece silver-plated woven fabric is cut into certain width through laser cutting.

### Encapsulation Layers

Two encapsulation methods are used including directly encapsulated with the commercial PU tape and encapsulated by two layers of silicon rubber using silicon rubber adhesive. The commercial PU tape is provided by Honsmed. The silicone rubber is part of the Ecoflex series from Smooth-On. The silicon rubber adhesive, also known as Sil-Poxy, is also provided by Smooth-On and it could form a strong while still flexible bonding between silicon parts, which ensures the stretchability of the encapsulated hydrogel-based strain sensor.

The formed sensors show high sensitivity of GF≥10 and fast recovery of less than 50 ms since the interaction between the hydrogel and the ions ensures good ionic conduction and high sensitivity while the CNT/graphene/MXene conductive agents to ensures electron conduction for resistance fast recovery—fast signal response. The double cross-linked polymer network creates both high stretchability and toughness which creates durability while chemical cross-linked networks maintain the structure of the hydrogel. The physical cross-linking networks maintain water content while introducing hydrogen bonds that reversibly break during stretching and enable good stretchability of the hydrogel.

The silver-plated woven fabric electrodes possess remarkable flexibility, high conductivity, and resistance to oxidation when in contact with the hydrogel, ensuring its seamless integration with the hydrogel.

PU tape or silicon rubber, known for their good biocompatibility, can directly interface with the skin, enabling the provision of ultra-thin encapsulation layers for the hydrogel to prevent dehydration of the hydrogel and prolong its service life.

During stretching, alterations in resistivity and conductivity arise from the rearrangement of the hydrogel's structure and the movement of ions within the conductive fillers. These changes facilitate the detection of subtle forces on the skin.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

The foregoing description of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention for various embodiments and with various modifications that are suited to the particular use contemplated.

1. A dual mode conductive hydrogel-based strain sensor having both an ion conductive mechanism and electron conductive fillers, the hydrogel-based strain sensor comprising:
a hydrogel including:
a first crosslinked hydrogel-forming polymer network;
a second crosslinked hydrogel-forming polymer network, the second crosslinked hydrogel-forming polymer network interpenetrating with the first hydrogel-forming polymer network without crosslinking between the first and second hydrogel-forming polymer networks;
a water-based liquid entrained by the first and second crosslinked hydrogel-forming polymer networks in an amount of approximately 50-75 wt. % of the hydrogel, the water including an ionically-conducting salt in an amount of 5-25 wt. % the formed hydrogel;
conductive fillers selected from two or more of graphene, carbon nanotube, and MXene; and
stretchable conductive electrodes formed on the hydrogel, the stretchable conductive electrodes selected from conductive particle-filled elastomers, stretchable metal meshes, and stretchable conductive fabrics.

2. The dual mode conductive hydrogel-based strain sensor of claim 1, wherein the first hydrogel-forming crosslinked polymer network includes a polyvinyl alcohol-based polymer and the second hydrogel-forming crosslinked polymer network includes an acrylamide-based polymer or a urethane-based polymer.

3. The dual mode conductive hydrogel-based strain sensor of claim 1, wherein the salt is selected from NaCl, CaCl₂, LiCl, or KCl.

4. The dual mode conductive hydrogel-based strain sensor of claim 1, further comprising a protective layer formed over the hydrogel.

5. The dual mode conductive hydrogel-based strain sensor of claim 4, wherein the protective layer is selected from silicone or polyurethane.

6. A strain measure measurement system comprising the dual mode conductive hydrogel-based strain sensor of claim 1 and a wearable flexible strain measurement device connected to the dual mode conductive hydrogel-based strain sensor.

7. A method for making the dual mode conductive hydrogel-based strain sensor of claim 1, comprising:
mixing a water-soluble synthetic polymer for the first hydrogel-forming polymer network, a polymerizable monomer for forming the second hydrogel-forming polymer network, and an ion conductive salt solution into a first mixture;
adding the electron conductive filler to the first mixture;
adding a crosslinking agent to the first mixture to crosslink the polymerizable monomer, creating the hydrogel having an interpenetrating network of the first hydrogel-forming polymer network and the second hydrogel-forming polymer network;
cutting a sensor blank from the hydrogel; and
forming electrodes on the sensor blank.

8. The method for making the dual mode conductive hydrogel-based strain sensor of claim 7, further comprising adding an initiator to form the second hydrogel-forming polymer network.

9. The method for making the dual mode conductive hydrogel-based strain sensor of claim 8, wherein the mixture includes:
5-15 wt % of the water-soluble polymer for forming the first hydrogel-forming crosslinked polymer network;
5-20 wt % of the polymerizable monomer;
5-25 wt % of salt, contributing to the ion conduction,
0.005-3 wt % of the electron conductive filler;
the crosslinking agent;
the initiator for a sol-gel process;
an accelerator for a sol-gel process; and
50-75 wt % of water.

10. The method for making the dual mode conductive hydrogel-based strain sensor of claim 9, wherein the crosslinking agent is N,N′-methylenebisacrylamide.

11. The method for making the dual mode conductive hydrogel-based strain sensor of claim 9, wherein the initiator is ammonium persulfate.

12. The method for making the dual mode conductive hydrogel-based strain sensor of claim 9, wherein the accelerator is N,N,N′, N′-tetramethylethylenediamine.
